(19) United States
(12) Patent Application Publication
(10) Pub. No.: US 2003/0149317 A1
(43) Pub. Date: Aug. 7, 2003

(54) Hydrogenation catalysts and methods

(76) Inventor: David Deck Rendina, North Vancouver (CA)

Correspondence Address: DAVID DECK RENDINA, 967 HERITAGE BLVD., NORTH VANCOUVER, BC, V7J 3G6, CA

(21) Appl. No.: 10/357,628

(22) Filed: Feb. 4, 2003

Related U.S. Application Data
(60) Provisional application No. 60/354,185, filed on Feb. 4, 2002.

(51) Int. Cl. C10G 45/00; C07C 5/02

(52) U.S. Cl. 585/250; 502/219; 208/143

(57) ABSTRACT

Catalysts and composite materials comprised of catalytically active materials intercalated with alkali metals and/or optionally coated on alkali metals or combinations of alkali and alkali earth metals are disclosed. Also disclosed are methods for enhancing reactions between hydrogen and organic materials by reacting said catalysts with solvents where such reactions generate a portion of the hydrogen and heat necessary to cause the desired reaction between an organic material and hydrogen and also act to fracture said catalytically active material into higher surface area particles with enhanced catalytic ability. Said catalysts may be dispersed in immiscible aprotic solvents to enhance transportation and safety considerations prior to use.

### RELATED REFERENCES

Reference is hereby made to commonly assigned provisional U.S. Patent application MATERIALS, METHODS AND SYSTEMS USEFUL IN THE UPGRADING OF HYDROCARBONS AND REDUCED PRODUCTION OF GHG, serial No. 60/354185, filed Feb. 4, 2002, the benefit of which is hereby claimed and the disclosure of which is incorporated herein by reference.

### FIELD OF THE INVENTION

The present invention relates to catalysts, composite materials, and new methods useful in the hydrogenation of organic materials.

### BACKGROUND OF THE INVENTION

New catalysts and new processes that enhance reactions between hydrogen and organic materials will be of great benefit to many industries. One industry that clearly exemplifies some of those benefits is the energy industry. The expanding need for energy in North America, combined with the depletion of known crude oil reserves, has created a serious need for the development of alternatives to crude oil as an energy source. One of the most abundant energy sources, particularly in the United States, is coal. Estimates have been made which indicate that the United States has enough coal to satisfy its energy needs for the next two hundred years. While Canada has over 330 billion barrels of oil bound in the bitumen tar sands of Alberta.

Unfortunately both coal and tar sands (bitumen) are solids, or nearly solids, at ambient temperatures. They have a high carbon content but hydrogen contents of typically only 5% to 9%. In comparison with fuels that are liquid at ambient temperatures, they are inconvenient to handle and unsuited to some applications. Most notably they cannot be used directly to fuel the internal combustion engines and turbines that dominate transportation infrastructures worldwide. Transportation fuels are derived overwhelmingly from crude oil, which has about twice the hydrogen content of coal. The hydrogen content of typical transportation fuels varies from ˜12.5% in some gasoline's to ˜14.5% in aviation turbine fuels. For coal or bitumen to replace them, they must be converted to liquids with similar hydrogen content.

Liquid fuels have long been produced from coal and bitumen. In general, current processes achieve this by either removing carbon (pyrolysis)/(coking) or adding hydrogen (liquefaction)/(hydrogenation). Since a comparison of the relative costs of crude and these other hydrocarbons are favorable, their commercial viability as transportation fuels depends on the overall economics of the conversion processes. Recent estimates (1990) indicate that two-stage conversion of coal to liquids has a product cost of about $38 per barrel and that the improved quality of the liquids makes them equivalent to oil costing $33 per barrel. Environmental costs are also high. Converting coal to transportation fuels reportedly results in 7-10 times as much CO₂ emissions as converting crude oil. This increase in CO₂ emissions at the processing stage has the effect of raising overall CO₂ emissions from the transportation sector by ˜50%, compared with transport based on conventional, refined petroleum products.

Incremental improvements to the established process steps are unlikely to decrease processing costs sufficiently to achieve a competitive price of $25 per barrel or to significantly reduce CO₂ emissions.

Structurally, bituminous coal typically consists of mono-cyclic and condensed aromatic rings, varying in size from a single ring to perhaps four or five rings, linked to each other by connecting bridges which are typically short aliphatic chains or etheric linkages. Generally, coal liquefaction and bitumen hydrogenation processes occur at temperatures exceeding 400 C. by rupturing the connecting bridges to form free radicals. The free radicals are then capped by a small entity such as hydrogen. If the free radicals are not capped, they will combine in condensation or polymerization reactions to produce large structures that are solid at room temperature.

The direct coal liquefaction technologies produce large amounts of hydrocarbon gases—ratios of liquids to hydrocarbon gasses usually being of the order of 3/1 to 4/1. Residence times of materials (reactants plus products) in the temperature zone above 350 C. are characteristically between 15 minutes and 1 hour. Such long exposure of the primary liquid molecules to temperatures above 350 C. results in extensive thermal cracking, yielding hydrocarbon gasses. Since more than half of the gas formed is methane, this cracking results in a large consumption of hydrogen and significantly increases the cost of production.

Recent studies described by Wiser et al, in U.S. Pat. No. 5,783,065 have reportedly demonstrated an improved simultaneous process for direct liquefaction and hydrogenation in the presence of a catalyst that generates a high proportion of liquid hydrocarbon product. Part of the benefit taught by the Wiser process is claimed to be as a result of short reaction times limiting hydrocracking and thus producing a higher quantity of liquid product.

It would be beneficial if catalytic hydrogenation of organic materials could be accomplished under conditions where the organic materials were not exposed to temperatures in excess to those required for the desired reaction for excess periods of time.

Whatever the organic feedstock, it can be generally stated that the efficiency of the hydrogenation process is enhanced by employing materials and methods whereby the molecules of feedstock are brought together with hydrogen, under temperature and pressure conditions required to cause the substances to react in the presence of a fresh catalytically active surface. In other words the heat, hydrogen, and organic material must all come together at the same time on the surface of the catalyst. Processes that enhance the probability of these conditions occurring simultaneously would be generally anticipated to improve efficiency. The present inventor has discovered that these and other benefits may be realized by the application of new catalysts comprised of intercalation compounds in an improved process.

Intercalation compounds may be conceptualized as being comprised of two components, a host intercalated material (M), and a visiting insertion material or intercalate (X). The host intercalation materials may be defined as elements, naturally occurring intermetallic compounds, or synthetic structures that allow the reversible insertion of ions, atoms, or molecules of another material—the intercalate—within spaces in the host structure. The bonding of the intercalated material with the intercalate does not change the chemical properties of the intercalate. In other words lithium intercalated into a material remains essentially lithium—hydrogen intercalated within a host remains essentially hydrogen and each can typically be repeatedly withdrawn and reinserted without damage to the host. It is often desirable that the host material is dimensionally stable during repeated intercalations and de-intercalations.

Alkali metal intercalated compounds are well known commercially produced materials. They are particularly well recognized with respect to their use as both anode and cathode materials in lithium batteries. Processes for intercalating alkali metals do not form a part of the present invention and any known methods for said intercalation may be employed. Intercalation methods may be exemplified by U.S. Pat. No. 3,933,688, to Dines and U.S. Pat. No. 4,040,917 to Whittingham, and many other examples known to those skilled in the art.

Morrison et al in U.S. Pat. No. 4,822,590 and again in U.S. Pat. No. 5,072,886, both of which are incorporated in the present application by reference, disclose how layered or porous materials intercalated with alkali metals may be separated or fractured into higher surface area materials by immersing the alkali metal intercalated material in a liquid that generates a gas upon reaction with the alkali metal. It is suggested that said separated or fractured materials may be useful in catalysis, however we are not taught the benefit that may be achieved by employing the heat and hydrogen and fresh catalytically active surfaces generated by said reaction to enhance the hydrogenation of organic material.

### SUMMARY OF THE INVENTION

A catalyst of the form MX is disclosed. Where M is an intercalation host and X is an alkali metal intercalated within the host. It is preferred that M is a chalcogenide and X is lithium. It is more preferred that M is a transition metal dichalcogenide and most preferred that M is MoS₂ or WS₂.

Said catalyst may be coated onto the surface of a particle to produce a composite of the form MXY. Where Y is comprised of Group IA-alkali metals, alkali metal hydrides, Group IIA-alkali earth metals, alkali earth metal hydrides, Group IIIA-metals, metal hydrides, and alloys, combinations, or mixtures of said materials. It is preferred that the alkali metals be sodium and phosphorous, and their hydrides, and that the alkali earth metals be calcium and magnesium and their hydrides, and that the metals be aluminum, or aluminum hydride or compounds or mixtures of these materials. It is most preferred that Y is comprised of sodium. It is further preferred that Y have dimensions between 1 and 100 microns.

Said catalyst and composites may be dispersed in a liquid material selected on the basis of its ability to protect the catalyst and/or composite from unintentional reactions prior to use, and on the basis of the compatibility of the said liquid material with the organic material to be hydrogenated. In reactions involving the liquefaction of coal or the hydrogenation of bitumen it is preferred that the organic liquid dispersant be a liquid hydrocarbon. It is most preferred that the liquid be a mixture of hexane and pentane.

The present inventor has discovered that the exfoliation or fracturing of these alkali metal intercalation catalysts, by reaction with liquids that generate hydrogen gas, while said catalysts are immersed in liquids or slurries comprised of organic material, will hydrogenate said organic material under relatively mild conditions.

According to the methods of this invention, there are provided processes for hydrogenating organic materials that comprise; mixing the organic material to be hydrogenated, with water, and a catalyst of the form MX, or a composition of the form MXY, in a manner such that the water reacts with the X component of the catalyst, or alternatively the XY components of the composite, to produce both heat and hydrogen while simultaneously fracturing the M component of the catalyst or composite and thereby expose catalytically active sites on said M component to enhance the hydrogenation of said organic material.

While not wishing to be limited in scope, the following experiments are supplied to illustrate aspects of the process disclosed.

### EXPERIMENT 1

A 500 ml sample of bitumen, reported supplied from the Cold Lake region of Canada was placed in a 1 liter metal reactor under argon atmosphere. A sample of catalyst of the form MX comprised of 10 grams of lithium intercalated MoS₂ dispersed in 50 ml of hexane was mechanically mixed into the bitumen and reactions began immediately. The remaining volume of the container was filled with water under pressure while shaking and the container was sealed. The container was mechanically shaken for approximately ½ hour. Overall temperatures within the reactor never exceeded 100 C., although it is assumed that spot temperatures at reaction sites may have been significantly higher. The resulting product was tested for asphaltene content by standard methods and it was determined that a ˜22% reduction in asphaltene content had been achieved.

### EXPERIMENT 2

Samples were prepared in a manner identical to those described in experiment 1 with the exception that a composite of the form MXY, comprised of 2 grams of lithium intercalated MoS₂ coated onto the surface of 10 grams of sodium metal particles, having an average particle size of 10 micron, dispersed in a 50 ml mixture of hexane and transformer oil, supplied from Powertech Labs of Surrey British Columbia, replaced the MX catalysts described in experiment 1. The resulting product was tested for asphaltene content by standard methods and it was determined that a ˜30% reduction in asphaltene content had been achieved.

The following claims and their obvious equivalents are believed to define the true scope of the invention.

I claim:

1. A catalyst of the form MX. Where;
M is an intercalation host, and
X is an intercalate alkali metal.

2. The catalyst of claim 1 where M is a chalcogenide.

3. The catalyst of claim 1 where M is a transition metal dichalcogenide.

4. The catalyst of claim 1 where M is MoS₂ or WS₂.

5. The catalyst of claim 1 where X is lithium.

6. A dispersion of the catalyst of claim 1 in a liquid hydrocarbon.

7. A composite of the form MXY. Where;
M is an intercalation host
X is an intercalate alkali metal
Y is an material selected from the Group IA-alkali metals, alkali metal hydrides, Group IIA-alkali earth metals, alkali earth metal hydrides, Group IIIA-metals, metal hydrides, and alloys, combinations, or mixtures of said materials.

8. The composite of claim 7 where M is a chalcogenide.

9. The composite of claim 7 where M is a transition metal dichalcogenide.

10. The composite of claim 7 where M is MoS₂ or WS₂.

11. The composite of claim 7 where X is lithium.

12. The composite of claim 7 where Y is sodium

13. The composite of claim 7 where the size of particles of Y are in the range of 1 to 100 microns.

14. A dispersion of the composite of claim 7 in a liquid hydrocarbon.

15. A process for hydrogenating an organic material. Said process being comprised of the following steps;
a) mixing a slurry or liquid form of the said organic material with the catalyst of claim 1, and water, for sufficient time and in a manner such that the water reacts with the X component of the catalyst, to produce both heat and hydrogen while simultaneously fracturing the M component of the said catalyst and thereby exposing catalytically active sites on said M component to enhance the hydrogenation of said organic material.

16. A process for hydrogenating an organic material. Said process being comprised of the following steps;
a) mixing a slurry or liquid form of the said organic material with the composite of claim 7, and water, for sufficient time and in a manner such that the water reacts with the XY components of the composite, to produce both heat and hydrogen while simultaneously fracturing the M component of the said composite and thereby exposing catalytically active sites on said M component to enhance the hydrogenation of said organic material.
